(19) United States
(12) Patent Application Publication
(10) Pub. No.: US 2011/0112650 A1
(43) Pub. Date: May 12, 2011

(54) MODIFIED MODULAR JOINT PROSTHESIS COMPONENT

(76) Inventor: Michael A. Masini, Ann Arbor, MI (US)

(21) Appl. No.: 12/614,514

(22) Filed: Nov. 9, 2009

(51) Int. Cl. A61F 2/38; A61F 2/32

(52) U.S. Cl. 623/20.15; 623/20.32; 623/22.21

(57) ABSTRACT

A modified joint prosthesis component is provided to reduce the backside wear of the joint implant. Whether the joint implant is for the knee joint, the hip joint or another joint, it is the modified bearing insert engaging surface of the metal base construct that provides for greater friction and therefore less micro-motion and resultant minimized backside wear.

### CROSS-REFERENCE TO RELATED APPLICATION

None.

### BACKGROUND OF THE INVENTION

The present invention relates generally to joint prostheses and more specifically to a joint prosthesis having a modified modular component to reduce the backside wear thereof. The component is primarily intended to be applicable for knees and hips but is envisioned to also apply to other joint surfaces.

Damage to joints caused by age, trauma, arthritis, osteoarthritis, and/or other disease related issues are becoming increasingly common. Orthopedic implants relieve patients of suffering from these conditions that effect the proper functioning of knee, hip, shoulder, and other joints. These implants are part of the total or partial joint replacement procedures that involve removal of the damaged parts of the relevant joint and replacing them. During such procedures, implant components especially selected to match the patient's needs are determined and implanted in the bones forming the joint.

Such components, whether for a total replacement or for a revision, are nearly uniformly of the modular variety. This is advantageous to the surgeon in that it allows intraoperative choice with regard to component thickness and implant constraint. During revision knee surgery in particular, modularity allows for the exchange of worn polyethylene inserts without disturbing the bony fixation of the metallic tray. Additionally, modularity also provides the surgeon with access to the fixation surface so that adjuvant screw fixation can be used before inserting the polyethylene.

These advantages, however, come at a price. For example, the additional interface between a tibial (for example) polyethylene insert and the underlying superior surface of the metallic tray creates an unintentional bearing surface. Micro motion is inevitable at this interface, therefore creating a source of polyethylene wear debris. This issue of “backside wear” cannot be mistaken for the normal wear issues relating to the main articulating surfaces of the implant, which, for the purposes of the present invention, will not be addressed. Backside wear is instead the sliding motions in the junction between the polyethylene bearing construct and the metal base construct that produces wear particles of polyethylene. Such particles can migrate out of the joint and into the body, and/or migrate into the interface between the polyethylene bearing construct and the metal base construct and scratch the metal base construct, particularly where the metal base construct is formed out of titanium.

Several different attempts have been made to try to reduce this so-called backside wear. Among them include: i) improving the lock between the metal and the polyethylene; ii) polishing the mating surfaces; iii) shaping the components; and iv) making the component out of two separate metals. First, improved locking mechanisms, by which the polyethylene is attached to the metal, via pins, plugs, dove-tails, grooves and other such mechanisms have been employed. However, the forces associated with the load bearing of these joints tend to relatively easily loosen these locking mechanisms thereby creating even greater problems. Next, polishing the mating surfaces lowers the coefficient of friction and therefore allows more motion, not less, and with more motion comes more wear. Next, manipulating the design and/or shape of the component parts of the implant, while perhaps lessening backside wear, more often than not results in a less than optimally functioning and subsequently failing prosthesis. Finally, it has been envisioned that by combining the different material characteristics of two different metals in the metal base construct, it may be possible to simultaneously form a superior bone-engaging face and a superior polyethylene-engaging face. Moreover, by selecting two appropriate metals for the metal base construct, superior bone ingrowth can be achieved while still avoiding the backside wear. Unfortunately, for reasons not yet certain, this attempt also fails to alleviate the issue.

While each of the aforementioned attempts has shown signs of success on particular individual applications, the issue of backside wear remains nevertheless fairly prevalent, and therefore needs to be addressed. The present invention addresses this issue by treating the surface of the metal intended to mate against the backside of the polyethylene. A process to treat, coat or otherwise roughen the surface will help prevent backside wear by increasing the coefficient of friction, introducing more friction and thereby reducing movement.

Accordingly, it is a general object of the present invention to provide for a treated modular component intended to mate against the backside of the polyethylene of an implant prosthesis.

It is a further object of the present invention to overcome the unsolved challenge of preventing and/or reducing backside wear of implant prostheses.

It is another object of the present invention to provide a modified modular joint prosthesis component that reduces movement by increasing friction.

It is yet another object of the present invention to apply the modified modular component to any joint prosthesis which otherwise produces wear particulate on its backside.

These and other objects, features and advantages of the present invention will be clearly understood through a consideration of the following detailed description and drawings. What follows is a preferred embodiment of the present invention. To assess the full scope of the invention, it is the claims that should be looked to, as the preferred embodiment is not the only embodiment within the scope of the invention.

### SUMMARY OF THE INVENTION

According to the present invention, there is provided a modified joint prosthesis having a metal base construct and a bearing insert construct. The bearing insert construct having a metal base construct engaging surface and an articulating surface, while the metal base construct has a bone engaging surface and an insert engaging surface. The insert engaging surface is roughened for engagement with the bearing insert.

There is also provided a modified modular joint prosthesis for use with an articulating joint component having a metal base component and a joint bearing component. The metal base component is inserted within bone and has an opposite bearing engaging surface, while the joint bearing component is positioned between the articulating joint component and the metal base component. The metal base component is treated on its bearing engagement surface so as to provide an increased coefficient of friction between the base and bearing components.

### BRIEF DESCRIPTION OF THE DRAWINGS

Although the characteristic features of this invention will be particularly pointed out in the claims, the invention itself, and the manner in which it may be made and used, may be better understood by referring to the following description taken in connection with the accompanying drawings forming a part hereof, wherein like reference numerals refer to like parts throughout the several views and in which:

FIG. 1 is a perspective exploded view of a prior art total knee replacement including a femoral component, a tibial baseplate and a corresponding tibial insert;

FIG. 2 is a cross-sectional exploded view of a prior art total hip replacement including a femoral component, an acetabular cup and a corresponding acetabular insert;

FIG. 3 is a frontal exploded view of a tibial insert and baseplate incorporating the principles of the present invention;

FIG. 4 is a frontal exploded view of a tibial insert and baseplate incorporating an alternative embodiment of the principles of the present invention of FIG. 3;

FIG. 5 is a frontal exploded view of a tibial insert and baseplate incorporating an alternative embodiment of the principles of the present invention of FIG. 3; and

FIG. 6 is a cross-sectional exploded view of an acetabular insert and acetabular cup incorporating the principles of the present invention.

### DESCRIPTION OF THE PREFERRED EMBODIMENTS

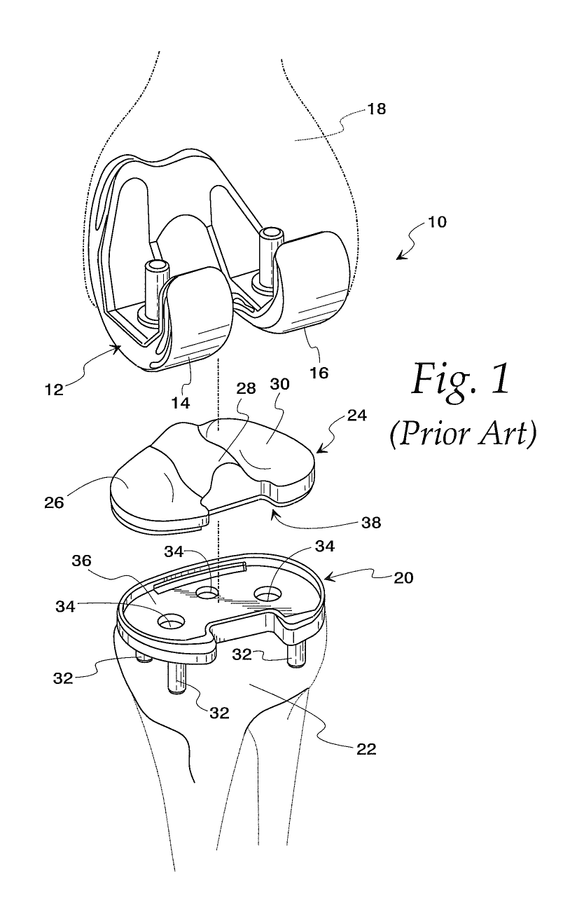

Referring now to the Figures, and in particular FIG. 1, the aforementioned prior art total knee replacement 10 is observed from behind the right knee and consists of a femoral component 12 having a medial condyle 14 and a lateral condyle 16. Femoral component 12 is surgically attached to the femur 18 and operates in conjunction with the tibial baseplate 20 attached to the tibia 22 and the insert 24 mounted to the baseplate 20.

The insert of a typical replacement is divided into three areas, the medial condyle compartment 26, the tibial eminence 28, and the lateral condyle compartment 30. Condyle compartments 26 and 30 have substantially similar concave geometrics and form the articulating surface for medial condyle 14 and lateral condyle 16 of the femoral component 12.

The tibial baseplate 20 is implanted by the surgeon onto the resected proximal tibia 22. One way to attach the baseplate 20 is to drill holes in the tibia 22 and insert the baseplate peg(s) 32 therein; another may be to place screws through the holes 34 of the baseplate 20 and into the tibia 22. Other means to affix the bone engaging face of the baseplate 20 to the tibia 22 have also been utilized in the art including both cemented and cementless fixation with or without bone ingrowth and ongrowth surfaces as well as osteoinductive coatings.

The insert 24 is then snap-locked onto the smooth top surface 36 of the baseplate 20. However the insert 24 may be coupled to the baseplate 20, whether snap-locked, glued, screwed, or otherwise, it is the backwide wear of the underside 38 of the polyethylene insert 24 against the baseplate 20 top surface 36 (or the superior polyethylene engaging face) which is the focus of the present invention.

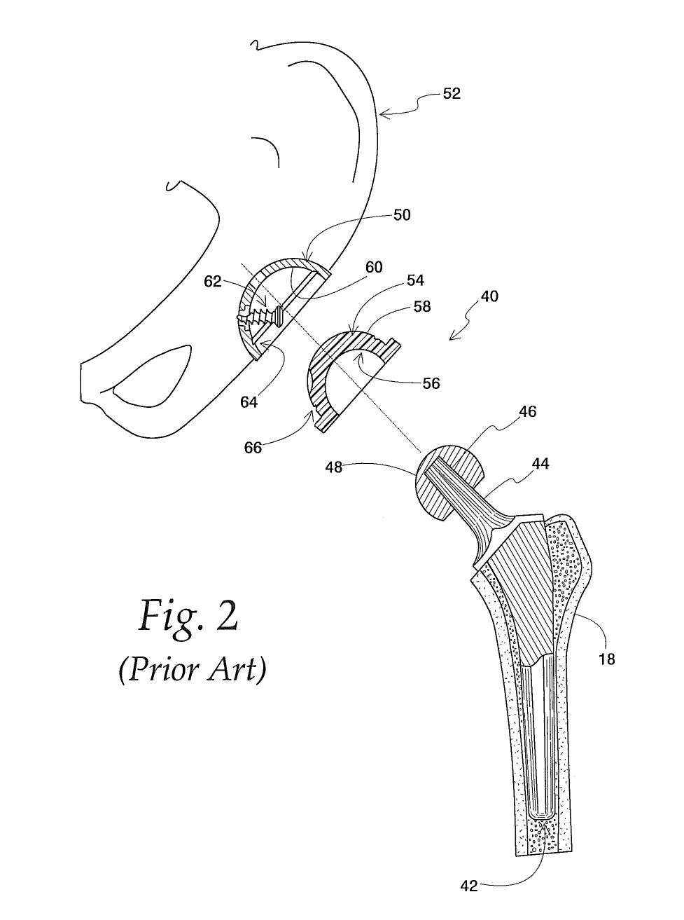

Such backside wear is also prevalent within other joint prostheses, including hip prostheses. For example, FIG. 2 illustrates a prior art total hip replacement 40 consisting of a femoral stem component 42 having a neck 44 with a tapered end 46 (or Morse taper) to interlock with the appropriately sized femoral head 48. The femoral stem 42 is surgically attached to the femur 18 and the femoral head 48 operates in conjunction with the acetabular cup 50 attached to the pelvic bone 52 and the insert 54 mounted to the acetabular cup 50.

The insert 54, like the knee insert of FIG. 1, includes an articulating surface 56 and a backside surface 58. The articulating surface 56 of the insert 54 mates with the femoral head 48 of the femoral stem component 42, while the backside surface 58 mates with the top surface 60 of the acetabular cup 50.

The acetabular cup 50 is implanted by the surgeon into the reamed portion of the pelvis. One way to attach the cup 50 is to drill holes in the pelvis and use screws 62. A plethora of holes and screw patterns, as well as other means to affix the cup 50 to the pelvis have been utilized in the art.

The insert 54 is then snap locked via cooperating cup and insert snap locks (64, 66) onto the smooth surface 60 of the acetabular cup 50. Once more, however the insert 54 may be coupled to the acetabular cup 50, whether snap-locked, glued, screwed, or otherwise, it is the backside wear of the underside 58 of the polyethylene insert 54 against the cup 50 surface 60 which is the focus of the present invention.

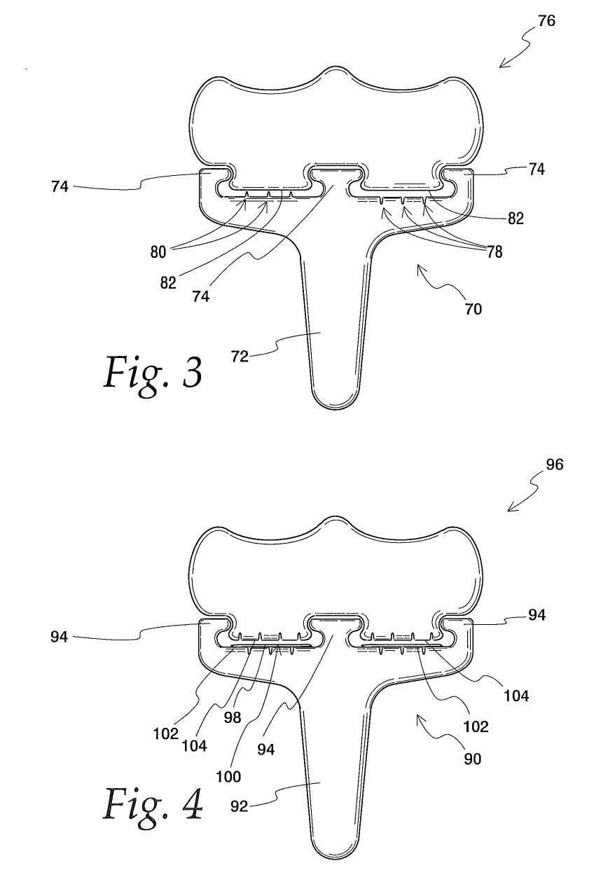

The approach of the present invention to address this backside wear problem differs from the previous four aforementioned prior art solutions. In particular it is envisioned that by increasing the coefficient of friction and introducing more friction to the backside of the polyethylene insert, movement and therefore wear will be reduced. Essentially, a roughened surface is created for direct attachment to the polyethylene. For example, the simplified frontal exploded view of the tibial insert and baseplate incorporating the principles of the present invention is shown in FIG. 3. Specifically, this simplified illustration shows a tibial component 70 including a single post 72 for implanting with the tibia and cooperating locking mechanisms 74 to aid in the attachment of the polyethylene tibial insert 76. The roughened surface 78 of the baseplate is this example has been created through the use of depressions such as laser etching grooves, dimples, pits, etc. Alternatively and/or cooperatively, protrusions (i.e. spikes, etc.) 80 may also be used. Perhaps such type of positive surfaces may be applied by a plasma spray or other coatings. In any event, it is this roughened surface 78 of the baseplate against the metal engaging surface 82 of the insert that minimizes wear by increasing the coefficient of friction and minimizing micro motion.

Another example incorporating the principles of the present invention is shown in FIG. 4. Specifically, this simplified illustration shows a tibial component 90 including a single post 92 for implanting within the tibia and cooperating locking mechanisms 94 to aid in the attachment of the polyethylene tibial insert 96. The increased friction here is due to bonding agents 98 such as permabond, PMMA, etc., or actual glues which would bond the polyethylene insert 96 to the metal shell of the tibial component 90. These agents may be strengthened through cooperating dimples, pits, etc. 100 on either or both of the tibial component 90 and/or the polyethylene tibial insert 96. It is the bonded surfaces of the baseplate 102 against the insert 104 that minimizes wear by increasing the coefficient of friction and minimizing micro motion.

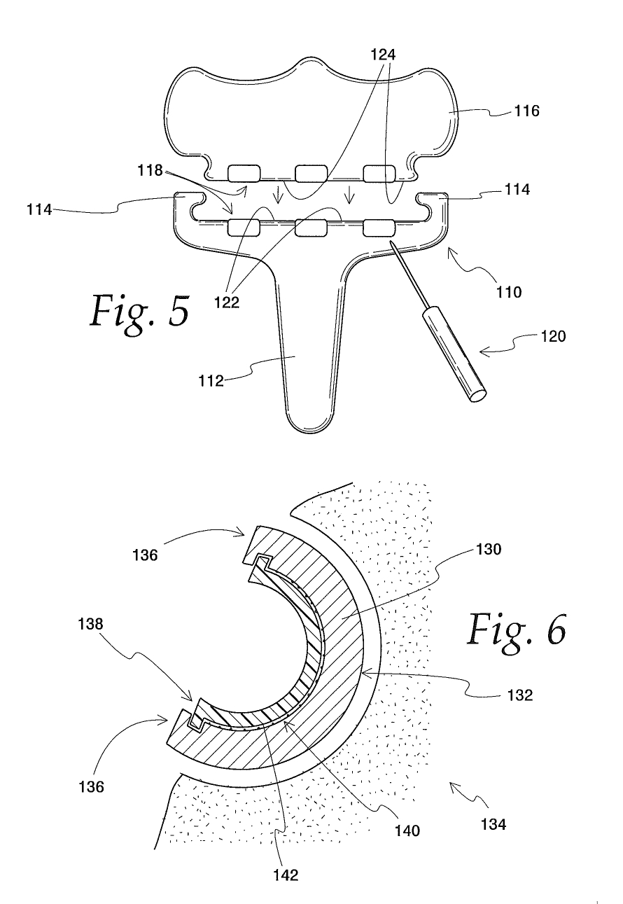

Another example incorporating the principles of the present invention as used with a knee joint implant is shown in FIG. 5. Specifically, this simplified illustration shows a tibial component 110 including a single post 112 for implanting within the tibia and cooperating locking mechanisms 114 to aid in the attachment of the polyethylene tibial insert 116. The increased friction here is due to certain portions of the surfaces having a state changing material such as polyetheretherketone or PEEK. In particular, the PEEK 118 could be embedded in both the metal surface of the tibial component as well as the polyethylene insert 116. The state changing material could be virtually any material that changes state and becomes a liquid with the application of vibration through an ultrasonic probe or the like. In any event, the PEEK (for example) is designed such that when an ultrasonic probe 120 is applied, and the energy is applied intraoperatively, the PEEK goes through a change in phase and becomes more liquid, and when ultrasound is stopped, it becomes more solid thus forming a bond between the polyethylene insert 116 and the tibial insert 110. It is the formation of this bond between the baseplate surface 122 and the metal engaging surface 124 of the insert that minimizes wear by increasing the coefficient of friction and minimizing micro motion.

It will be understood that the principles of the present invention are not limited to knee joint implants. In fact, it is envisioned that any joint implant that produces backside wear can be improved with these concepts. For example, a simplified cross-sectional view of an acetabular insert and an acetabular cup as used in a hip implant is shown in FIG. 6. Specifically, this illustration shows an acetabular metal cup 130 including a metal ingrowth (or outgrowth) surface 132 for implanting within the reamed out pelvis bone 134 and cooperating locking mechanisms 136 to aid in the attachment of the polyethylene acetabular insert 138. The plasma spray, spiked or laser indented surface 140 of the metal cup 130 will reduce liner motion and backside wear. More particularly, it is the treated surface 140 of the metal cup against the inserts metal engaging surface 142 that minimizes wear by increasing the coefficient of friction and minimizing micro motion.

Whether the present invention is utilized on a knee joint implant, a hip joint implant or some other joint implant, the unique material properties of polyethylene (the currently most widely used insert biomaterial) provides an additional advantage. In particular, polyethylene possesses a unique material property which allows it to actually deform without failing. This so-called CREEP happens under load and over time. For example, if there are screw holes beneath the polyethylene, the polyethylene itself will actually deform and project into the screw holes. Such CREEP adds strength to the bond between the polyethylene and the base metal surface. This material property is particularly advantageous in newer polyethylene's which employ cross-linking to reduce wear and exhibit enhanced creep over older polyethylene products.

Accordingly, rather than making the polyethylene contact surface of the metal base construct smoother, and decrease the coefficient of friction as discussed in the prior art, the concept discussed herein takes the opposite approach and increase the coefficient of friction. In particular, if the metal base construct surface that contacts the polyethylene bearing construct had more friction (not less), then the micro motion between the two and therefore the “backside wear” of the polyethylene between these mating surfaces would be minimized. While this disclosure describes a number of specific ways to increase this friction, such as through a roughened surface, pits, grooves, etchings, plasma spray or other coatings, bonding agents state changing materials, etc., it will be understood that the invention is not limited thereto. It is the concept of increasing the friction between the polyethylene insert and the base metal surface, and not necessarily how the friction is increased, that is the inventive aspect.

1. A modified modular joint prosthesis, comprising:
a metal base construct having a bone engaging surface and an insert engaging surface;
a bearing insert construct have a metal base construct engaging surface and an articulating surface; and
said insert engaging surface of said metal base construct having a roughened surface for engagement against said metal base construct engaging surface of said bearing insert.

2. A modified modular joint prosthesis of claim 1 wherein said metal base construct is a tibial baseplate.

3. A modified modular joint prosthesis of claim 1 wherein said metal base construct is an acetabular cup.

4. A modified modular joint prosthesis of claim 1 wherein said bearing insert construct is polyethylene.

5. A modified modular joint prosthesis of claim 1 wherein said metal base construct and said bearing insert construct have cooperating locking mechanisms.

6. A modified modular joint prosthesis of claim 1 wherein said insert engaging surface of said metal base construct is laser etched.

7. A modified modular joint prosthesis of claim 1 wherein said insert engaging surface of said metal base construct is spray coated.

8. A modified modular joint prosthesis of claim 1 wherein said insert engaging surface of said metal base construct is treated with a bonding agent.

9. A modified modular joint prosthesis of claim 1 wherein said insert engaging surface of said metal base construct is treated with a state changing material.

10. A modified modular joint prosthesis of claim 9 wherein said state changing material is altered with an ultrasonic probe.

11. A modified joint prosthesis for use with an articulating joint component, the prosthesis comprising:
a metal base component for insertion within bone, said base component having a bearing engaging surface;
a joint bearing component positioned between said articulating joint component and said metal base component; and
said base component being treated on said surface to have an increased coefficient of friction between said base component and said bearing component.

12. The prosthesis as defined in claim 11 wherein said metal base component is a tibial baseplate.

13. The prosthesis as defined in claim 11 wherein said metal base component is an acetabular cup.

14. The prosthesis as defined in claim 11 wherein said bearing component is polyethylene.

15. The prosthesis as defined in claim 11 wherein said metal base component and said bearing component have cooperating locking mechanisms.

16. The prosthesis as defined in claim 11 wherein said bearing engaging surface of said base component is laser etched.

17. The prosthesis as defined in claim 11 wherein said bearing engaging surface of said base is spray coated.

18. The prosthesis as defined in claim 11 wherein said bearing engaging surface of said base is treated with a bonding agent.

19. The prosthesis as defined in claim 11 wherein said bearing engaging surface of said base is treated with a state changing material.

20. The prosthesis as defined in claim 19 wherein said state changing material is altered with an ultrasonic probe.

21. The prosthesis of claim 1 wherein the roughened surface is comprised of one or more depressions.

22. The prosthesis of claim 21 wherein the depression is a groove.

23. The prosthesis of claim 21 wherein the depression is a pit.
